(19) United States
(12) Patent Application Publication
(10) Pub. No.: US 2007/0258852 A1
(43) Pub. Date: Nov. 8, 2007

(54) Disinfected Elevator Passenger Interface

(75) Inventors: Norbert Hootsmans, South Glastonbury, CT (US); Alberto Vecchiotti, Middletown, CT (US); Pei-Yuan Peng, Manchester, CT (US); Jae-Hyuk Oh, Tolland, CT (US); John M. Milton-Benoit, East Hartford, CT (US)

Correspondence Address: CARLSON GASKEY & OLDS, 400 W MAPLE STE 350, BIRMINGHAM, MI, 48009, US

(73) Assignee: OTIS ELEVATOR COMPANY, Farmington, CT (US)

(21) Appl. No.: 11/574,102

(22) PCT Filed: Sep. 20, 2004
(86) PCT No.: PCT/US04/30655

§ 371 Date: Feb. 22, 2007

(51) Int. Cl. A61L 2/10; A61L 2/08; B01J 19/08; B01J 19/12

(52) U.S. Cl. 422/24; 422/186; 422/186.3; 422/22

(57) ABSTRACT

A passenger interface device (20) includes at least one input member (22, 24, 26) having a contact surface (28) that is adapted to be touched by an individual. A disinfectant (30) is on the contact surface (28). The disinfectant (30) comprises a radiation-activated material. A source of radiation (32) irradiates the disinfectant (30) to disinfect the contact surface of the passenger interface device. In one example, titanium dioxide is used as a photocatalyst that disinfects the contact surface responsive to ultraviolet light radiation.

### FIELD OF THE INVENTION

This invention generally relates to elevator systems. More particularly, this invention relates to a passenger interface for an elevator system.

### DESCRIPTION OF THE RELATED ART

Elevator system are well know in widespread use. Passenger interfaces allow individuals to place a call to be carried to a desired destination. For example, hall call buttons allow an individual to provide an indication whether they desire to travel up or down from a building level. Typical hall call button arrangements have an up and down button in an elevator lobby or near an elevator entrance.

Other passenger interfaces include car operating panels inside of elevator cars. Typical car operating panels include a plurality of buttons that can be pressed to indicate a desired destination floor.

Other elevator systems include destination entry devices located outside of an elevator car that allow an individual to provide an indication of their desired destination before entering an elevator car. Such devices work in a known manner.

One issue presented by elevator passenger interface devices is that many people touch them through the course of a day. With communicable diseases, elevator passenger interfaces may present an opportunity for one passenger to contract an illness from another passenger. This became a particular concern with the recent SARS situation in Asia, for example. People were becoming hesitant to press elevator call buttons, for fear of contracting SARS.

One solution includes cleaning elevator call buttons on an hourly basis to alleviate concerns for individuals needing to access those buttons. This approach obviously introduces additional labor and expense for building owners, for example.

Another proposal is discussed in the Japanese Patent Publication No. 11-306902. In that document, an antimicrobial film is adhesively secured onto a call button. Such a film must be periodically replaced, which introduces additional labor and expenses.

It would be beneficial to provide an arrangement for disinfecting an elevator passenger interface in a cost saving and effective manner. This invention address that need.

### SUMMARY OF THE INVENTION

An example passenger interface device designed according to this invention includes an input member that has a contact surface that is adapted to be touched by an individual. A radiation-activated disinfectant is on the contact surface. A source of radiation activates the disinfectant.

In one example, the disinfectant comprises a photocatalyst. In a particular example, the disinfectant comprises TiO₂. In some examples, the source or radiation comprises an ultraviolet light source. One disclosed example includes a laser radiation source.

A disclosed method of disinfecting a passenger interfacing includes irradiating a radiation-activated disinfectant on a passenger interface contact surface.

The various features and advantages of this invention will become apparent to those skilled in the art from the following detailed description. The drawings that accompany the detailed description can be briefly described as follows:

### BRIEF DESCRIPTION OF THE DRAWING

FIG. 1 schematically illustrates an example passenger interface device.

FIG. 2 schematically shows another example passenger interface device.

FIG. 3 schematically shows selected portions of the embodiment of FIG. 2.

### DETAILED DESCRIPTION OF THE PREFERRED EMBODIMENT

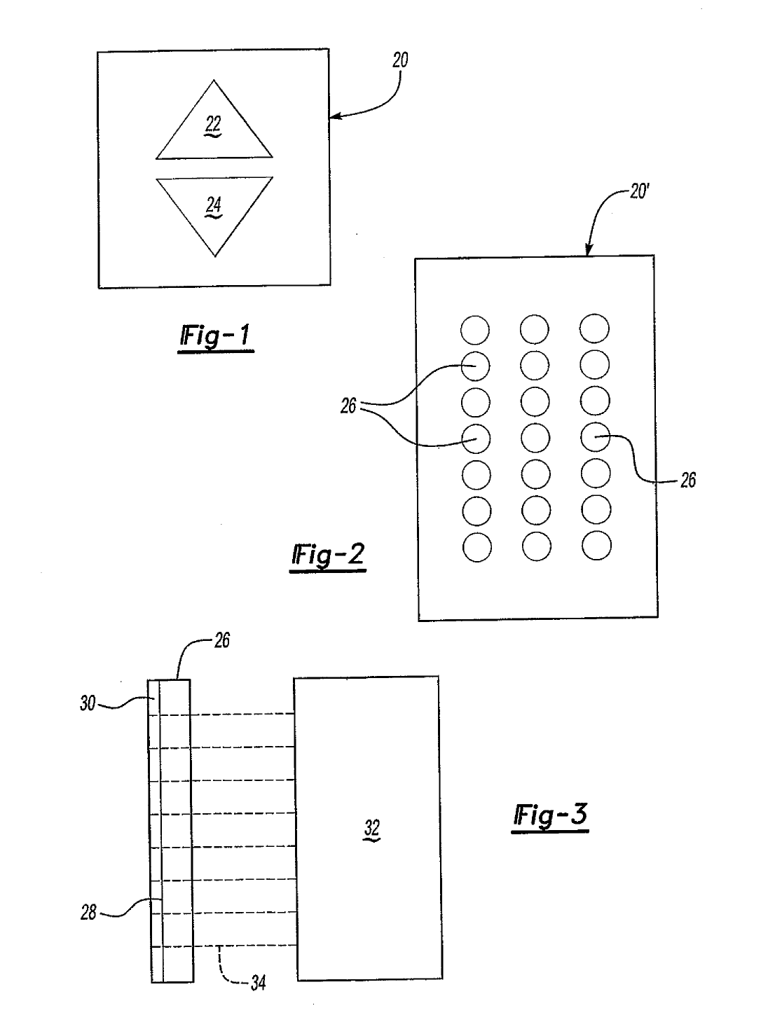

FIG. 1 schematically shows a passenger interface device 20 that is useful with an elevator system, for example. In this example, the passenger interface device 20 operates as hall call buttons to allow an individual to place a call for elevator car service. The illustrated example includes a first input member, which is an up button 22, and a second input member, which is a down button 24. When an individual presses either one of these, an elevator system responds in a known manner to send a car to eventually carry that individual to their intended destination.

In one example, the passenger interface device 20 comprises a configurable touch screen display as the input member. Such a display screen operates in a known manner to provide images of the buttons 22 and 24. When an appropriate portion of the screen is touched, that provides a signal to the elevator system to operate in a known manner.

FIG. 2 schematically shows another passenger interface device 20′. This example includes a plurality of input members 26, which correspond to buttons that indicate particular destinations (i.e. floor levels).

In one example, the passenger interface device 20′ comprises a car operating panel. The input members 26 in one example comprise physical buttons that can be pressed by an individual to provide an indication regarding their intended destination.

In another example, the car operating panel passenger interface device 20′ includes a configurable touch screen display that includes images of the buttons 26 as the input member. The touch screen display includes a screen that can be pressed by an individual to provide an indication of their intended destination.

In another example, the passenger interface device 20′ is part of a destination entry device located outside of an elevator car. In one example, the illustrated buttons 26 correspond to physical buttons that can be pressed by an individual to provide an appropriate indication of their intended destination. For example, the buttons 26 may be part of numerical keypad or may each represent a particular building level.

In another example, a destination-entry-style passenger interface device 20′ comprises a configurable touch screen display input member that operates in a known manner.

Regardless of the configuration of the passenger interface device, a contact surface that is adapted to be touched or pressed by an individual includes a radiation-activated disinfectant on the contact surface. FIG. 3 schematically shows one example arrangement where one of the buttons 26 has a contact surface 28. A radiation-activated disinfectant coating 30 is on the contact surface 28 for disinfecting the contact surface.

A source of radiation 32 irradiates the disinfectant coating 30 as schematically shown at 34. The source of radiation 32 in one example is positioned on an opposite side of the button 26 from the contact surface 28. In one example, the source of radiation 32 is positioned within the wall space for a hall call button passenger interface arrangement. In another example, the source of radiation 32 is housed within a car operating panel housing. In another example, the source of radiation 32 is housed in a kiosk that is part of a destination entry device.

In one example, the disinfectant coating 30 comprises a nano-photocatalyst. One example includes titanium dioxide (TiO₂). In this example, the photocatalyst is activated to provide disinfecting qualities responsive to ultraviolet light radiation. In such an arrangement, the source of radiation 32 comprises an ultraviolet light source. One example includes an ultraviolet light laser for specifically directing ultraviolet radiation toward the disinfectant coating 30 on the contact surface 28.

In examples, where the passenger interface includes physical buttons, the buttons preferably are made of a material that allows the radiation for activating the disinfectant coating 30 to penetrate to the coating if the source of radiation 32 is positioned on an opposite side of the button 26 from the contact surface 28. The coating 30 in such an example does not allow the radiation to pass through the coating and, therefore, prevents such radiation from proceeding into an area where an individual may be exposed to it.

Other radiation-activated disinfectant materials may be used on a passenger interface device. Those skilled in the art who have the benefit of this description will be able to select from among known materials which such properties to meet the needs of their particular situation.

The source of radiation 32 need not necessarily be positioned “behind” the contact surface of the button or the display panel but may be otherwise strategically positioned to irradiate the disinfectant coating 30 to provide the desired results.

The preceding description is exemplarily rather than limiting in nature. Though variations and modifications to the disclosed examples may become apparent to those skilled in the art that do not necessarily be part from the essence of the this invention. The scope of legal protection given to this invention can only be determined by studying the following claims.

1. A passenger interface device, comprising:
an input member having a contact surface that is adapted to be touched by an individual and a radiation-activated disinfectant coating on the contact surface; and
a source of radiation for activating the disinfectant.

2. The device of claim 1, wherein the disinfectant comprises a photocatalyst.

3. The device of claim 2, wherein the disinfectant comprises TiO₂.

4. The device of claim 1, wherein the source of radiation comprises an ultraviolet light source.

5. The device of claim 1, wherein the source of radiation comprises a laser.

6. The device of claim 1, wherein the input member comprises a display panel.

7. The device of claim 1, wherein the input member comprises a button.

8. The device of claim 7, wherein the button comprises an elevator hall call button.

9. The device of claim 7, wherein the button comprises an elevator car operating panel button.

10. The device of claim 7, wherein the input member comprises an elevator destination entry device button.

11. The device of claim 1, wherein the source of radiation is positioned on an opposite side of the input member from the contact surface.

12. The device of claim 11, wherein the input member comprises a material that allows at least some radiation from the source of radiation to pass through the material and the disinfectant prevents the radiation from passing through the disinfectant.

13. A method of disinfecting a passenger interface, comprising:
irradiating a radiation-activated disinfectant coating on a passenger interface contact surface.

14. The method of claim 13, including coating the contact surface with the disinfectant.

15. The method of claim 13, wherein the disinfectant comprises a photocatalyst.

16. The method of claim 15, wherein the disinfectant comprises TiO₂.

17. The method of claim 13, including irradiating the disinfectant with ultraviolet light.

18. The method of claim 13, wherein the passenger interface comprises one of an elevator hall call button, an elevator car operating panel button, an elevator destination entry device button or an elevator passenger input device display screen.
